(19) United States
(12) Patent Application Publication
(10) Pub. No.: US 2020/0008379 A1
(43) Pub. Date: Jan. 9, 2020

(54) MICROALGAE-BASED SOIL INOCULATING SYSTEM AND METHODS OF USE

(71) Applicant: MyLand Company, LLC, Phoenix, AZ (US)

(72) Inventors: Andrew D. Ayers, Chandler, AZ (US); Mark R. Edwards, Tempe, AZ (US); Dane Hague, Scottsdale, AZ (US); Robert Thompson, Scottsdale, AZ (US)

(21) Appl. No.: 16/534,907

(22) Filed: Aug. 7, 2019

Related U.S. Application Data
(63) Continuation-in-part of application No. 16/207,528, filed on Dec. 3, 2018, now abandoned.
Application No. 16/207,528 is a continuation-in-part of application No. 15/647,005, filed on Jul. 11, 2017.
Application No. 16/207,528 is a continuation of application No. 14/069,932, filed on Nov. 1, 2013, now Pat. No. 10,172,304.
Application No. 14/069,932 is a continuation of application No. PCT/US12/36293, filed on May 3, 2012.
(60) Provisional application No. 61/481,998, filed on May 3, 2011.

(51) Int. Cl. A01G 33/00; C12N 1/12; C12M 1/00; C12M 1/12; C05F 11/08; C05D 9/02

(52) U.S. Cl. CPC A01G 33/00; C12N 1/12; C12M 21/02; C12M 23/52; C05D 9/02; C12M 37/00; C12M 43/00; C05F 11/08; C12M 29/04

(57) ABSTRACT

Some embodiments include a microalgae culturing system including a bioreactor adapted to propagate microalgae in a culture solution using in combination at least one of natural and artificial light, and at least one nutrient including at least a carbon source, where the microalgae are freely suspended in and form part of the culture solution. A microalgae feed source is coupled to the bioreactor and a first controller between a water conditioning assembly and the bioreactor. The water conditioning assembly is coupled as an input of supply water to the bioreactor, and configured to condition the supply water to a specified purity that enables substantially unhindered growth of the microalgae in the culture solution to a specified concentration, and the first controller is configured to control supply of the microalgae feed source to the bioreactor.

### RELATED APPLICATIONS

This application is a continuation-in-part application of U.S. patent application Ser. No. 16/207,528, filed Dec. 3, 2018, entitled “Microalgae-Based Soil Inoculating System and Methods of Use”, which is a continuation of U.S. patent application Ser. No. 14/069,932, filed Nov. 1, 2013, entitled “Microalgae-Based Soil Inoculating System and Methods of Use”, now issued as U.S. Pat. No. 10,172,304, which is a continuation of International Patent Application No. PCT/US12/36293, filed May 3, 2012, entitled “Microalgae-Based Soil Inoculating System and Methods of Use”, which claims the benefit of and priority to U.S. Provisional Application No. 61/481,998, filed May 3, 2011, entitled “Microalgae-Based Soil Inoculating System and Methods of Use”, and further is a continuation-in-part of U.S. patent application Ser. No. 15/647,005, filed Jul. 11, 2017, entitled “Soil Enrichment Systems and Methods”, this application incorporates the disclosure of all such priority applications by reference. To the extent that the present disclosure conflicts with any referenced application, however, the present disclosure is to be given priority.

### BACKGROUND

Microbes in soil have many well-known beneficial effects. While there are many references to algae herein, such references are used solely as helpful examples, and do not limit the scope of the inventions described and claimed herein, which are directed to microbes generally as well.

Algae have the ability to adapt to their environment. For instance, algae found in soil in the Southwestern deserts have adapted to elevated temperatures, alkaline pH levels, and periods of desiccation, while algae in northern climates have adapted to much lower temperatures, freeze-thaw cycles, higher soil moisture levels, and more acidic soil pH levels, etc.

Endemic algae fill a niche in the field ecosystem. Within the soil ecosystem, a symbiosis with other organisms has developed resulting in a biochemical environment where compounds produced by the endemic algae may augment the growth of other desirable microbes and depress the growth of undesirable or non-beneficial organisms. For example, algae are known to produce biochemicals such as amino acids, hormones, peptides, and fatty acids that augment the growth of beneficial microorganisms. These beneficial biochemicals also directly help the crop plants. The beneficial microorganisms produce biochemicals that the algae and crop can utilize to grow (e.g., sugars and vitamins) resulting in continued algal and crop growth. At the same time, algae may produce compounds that are antibacterial, antifungal, algicidal, and/or antiprotozoal which prevent the growth of unwanted microbes in soil and surface waters.

When soil algae die, cellular biochemicals are released which can directly feed the soil biome and any crop plants growing in the soil. These biochemicals are large molecules (e.g., such as proteins, fats, dyes, peptides, nucleic acids, etc.), some or all of which can be absorbed by the crop plant, resulting in crops with greater nutritional value.

If live, foreign algae are introduced into the soil, the ecosystem is forced to rebalance. This imbalance can lead to the production of one or more unwanted biochemicals (such as a toxin), or the absence of an important biochemical which may be required by the crop plant.

When algae are introduced to the soil, the metabolic activity in the soil increases, resulting in greater CO₂ production. This is particularly true for live algae whose metabolic activities continue after introduction to the soil. This CO₂ production lowers the pH of the soil resulting in the dissolution of calcium and magnesium carbonate bonds, thereby opening the soil for greater root penetration and increased water and fertilizer movement. This increased water movement carries more salts out of the root zone, thereby reducing the osmolarity within the root zone, and increasing the bioavailability of macro and micronutrients to the crop. The lower pH also frees up bound potassium and phosphorus making it available to the plants. Algae excrete extracellular phosphatases almost immediately upon the onset of phosphorus limited conditions. These compounds release the phosphates from soil particles and make them available to the plants. Green algae also produce polysaccharides which hold onto water until it is needed.

Substantially constant or periodic addition of algae can result in a desirable buildup of organic matter (humus) within the soil which also has the property of holding water and nutrients which can be released to the plants as needed. Other methods of introducing humus to the soil generally require tilling-in of organic matter (compost, various plant cuttings, manures, etc.), which can best be performed when a field is between plantings. Humus aids in the formation of natural iron chelates (fulvic acids-Fe), which prevents soil from being blocked by calcium and magnesium carbonates, thus avoiding chlorosis problems induced by low bioavailability of these nutrients. Chlorosis is the reduction in the green color of plants due to a reduction in the amount of chlorophyll in the leaves brought on by a lack of bioavailable macro and micronutrients such as nitrogen (N), magnesium (Mg), calcium (Ca), and iron (Fe), even when these nutrients are present in the soils.

Ion exchange capacity is a quantitative means for describing the binding of fertilizer elements to soil particles for storage and release. Humus ion exchange capacity (e.g., 400 to 600 meq/100 g) is 5 to 10 times higher than that of clays (e.g., 50 to 150 meq/100 g). It is this capacity which allows the retention of fertilizers within the soil for use by the plants as needed. As plants utilize the nitrogen (N), phosphorus (P), and potassium (K) in the soil, the stored elements are released from the humus as needed. By combining with humic substances, copper and other trace elements become less toxic and more readily available to the plants.

Fertilizers are more effective if combined with microalgae. Algae cells process fertilizers by breaking down certain molecules into more bioavailable forms that plants can more readily use. The nutrients are then more efficiently and completely absorbable by the root system of the plants. For example, ammonium nitrate, an excellent source of nitrogen, is one of the most common bulk fertilizers used to grow crops. While plants can immediately absorb the nitrate in this fertilizer, the ammonium component is less accessible to the plant. Microalgae cells will absorb the ammonium, naturally convert it to nitrogenous biochemicals, and upon their death, will release these valuable biochemicals to the plant for easy consumption. Additionally, the nutrients from fertilizers can bind to the microalgae cells or their organic remains and are less likely to be lost in run-off water during rains or irrigation. Upon their death, the algae can also feed bacteria in the soil, which can convert the ammonium ion into nitrate ions.

Algae produce growth regulators (e.g., gibberellic acid) that improve salt tolerance, induce seed germination and increase plant growth rate and fruit production. Artificial or concentrated growth regulators are expensive, especially when applied in substantial amounts, making it impractical for growers to replicate this effect by use of other products.

Algae play a role in controlling agricultural pests by directly producing antibiotics and antifungal compounds, and by feeding the beneficial microbes in the soil which produce other pest fighting compounds. These compounds give the plants the ability to prevent the invasion of pathogenic species. Disease and pests are also resisted due to the improved vigor of the plants.

As discussed above, live microalgae cells can function as a catalyst to tap and utilize all of the benefits available from standard fertilizers; and also, to provide a natural supply of essential compounds and phytochemicals, while supporting the overall efficacy of the growing environment. These potent attributes work in concert to stimulate plants to grow heartier and more quickly; and to consistently produce a more abundant, higher quality and more nutrient rich end-product such as a crop. The benefits from an additive of microalgae cells are available when the algae cells that are delivered to the soil are in healthy living form and in great concentration. The selection and formulation of the algae additive is critical to its overall impact. When correctly instituted, a microalgae additive program is simple to manage, and offers breakthrough potential in agricultural production. The impact may be greatest in the most depleted soils such as arid soils that have significant salt and caliche buildup with minimal organic matter. Further, by selecting endemic algae for propagation and delivery to an agricultural production area, there is a higher survival rate, and a greater and faster impact on soil health.

### SUMMARY OF THE INVENTION

Some embodiments include a culturing system comprising a bioreactor adapted to propagate microalgae in a culture solution using in combination natural and/or artificial light, and at least one nutrient comprising at least a carbon source, where the microalgae are freely suspended in and form part of the culture solution. Some embodiments include an algae nutrient supply coupled to the bioreactor and a first controller between a water conditioning assembly and the bioreactor. In some embodiments, the water conditioning assembly is coupled as an input of supply water to the bioreactor, and configured to condition the supply water to a specified purity that enables substantially unhindered growth of the microalgae in the culture solution to a specified concentration. Further, in some embodiments, the first controller can be configured to control the delivery of the algae nutrient supply to the bioreactor. In some embodiments, a carbon dioxide source is coupled to the bioreactor, where the carbon dioxide is injected into the culture solution as the carbon source.

Some further embodiments include a second controller coupled to a probe and configured to regulate release of carbon dioxide from the carbon dioxide source to the bioreactor based at least in part on one or more measurements from the probe, where the carbon dioxide is injected into the culture solution as the carbon source.

In some embodiments, the probe is a pH probe configured to measure a pH of the culture solution. In some embodiments, the water conditioning assembly includes an ozone generator coupled to an ozone contactor, where the ozone generator is configured to generate ozone and deliver the ozone to at least partially ozonate the supply water.

Some embodiments include a solids filter downstream from an outlet of the ozone contactor, where the solids filter is configured to remove solids from ozonated supply water exiting the ozone contactor. Some embodiments include a carbon filter and/or a UV light system positioned downstream from the solids filter, where the carbon filter and/or the UV light system can at least partially de-ozonate the ozonated supply water.

Some embodiments include at least one pressurized air supply coupled to the bioreactor, where the at least one pressurized air supply can generate gas bubbles to at least partially aerate and/or agitate the culture solution. In some embodiments, the gas bubbles include CO₂, N₂, and/or O₂. Some embodiments further comprise at least one water reservoir or tank providing or coupled to the input of supply water.

Some further embodiments comprise a mobile trailer supporting at least the bioreactor, the water conditioning assembly, and the carbon dioxide source. In some embodiments, the microalgae feed source comprises a fertilizer, a macro-nutrient, a micro-nutrient, and at least two different microalgae species.

In some embodiments, the macro-nutrient is selected from the group consisting of phosphorus, nitrogen, carbon, silicon, calcium salt, magnesium salt, sodium salt, potassium salt, and sulfur; and the one or more micronutrients is selected from the group consisting of manganese, copper, zinc, cobalt, molybdenum, vitamins and trace elements. Further, in some embodiments, the micro-nutrient comprises a vitamin and a mineral added to the conditioned supply water.

Some embodiments comprise a telemetry system configured for a remote monitoring and/or controlling operation of one or more of the first controller, the second controller, the bioreactor, and at least one component or assembly of the water conditioning assembly.

In some embodiments, the artificial light comprises LED lights positioned within the bioreactor and/or proximate to a surface of the bioreactor and exposing the microalgae to light.

In some embodiments, the carbon dioxide source comprises a tank comprising carbon dioxide gas, and/or a carbon dioxide generator, and/or a carbon dioxide-sequester that sequesters and temporarily stores atmospheric carbon dioxide.

In some further embodiments, the microalgae feed source comprises a first algae type, and/or a second algae type, and/or bacteria, and/or fungi. Some embodiments further comprise a flow-imaging device coupled to an output of the bioreactor, where the flow imaging device is configured to create images of algae, predators, and contaminants in the culture solution for quality control monitoring. Some embodiments further comprise a microorganism mixer configured to blend algae, and/or bacteria, and/or fungi, with any of the culture solution exiting the bioreactor.

Some embodiments include a method comprising preparing one or more microbe-containing samples from at least one location of a current or planned plant growth area, and preparing at least one cultured sample by culturing microbes from the sample. Further, some embodiments include selecting at least one target species of microbe from the at least one cultured sample, and propagating the at least one selected target species of microbe to increase the concentration of the at least one target species of microbe in the at least one cultured sample. Some embodiments include providing a bioreactor adapted to propagate the at least one selected target species in a culture solution, where the at least one selected target species being freely suspended in and forming part of the culture solution. Further, some embodiments include coupling a feed source to the bioreactor and a first controller between a water conditioning assembly and the bioreactor, where the water conditioning assembly is coupled as an input of supply water to the bioreactor, and configured to condition the supply water to a specified purity that enables substantially unhindered growth of the at least one selected target species in the culture solution to a specified concentration. Further, in some embodiments, the first controller is configured to control supply of the feed source to the bioreactor. Further, in some embodiments of the method, a carbon dioxide source is coupled to the bioreactor.

Some embodiments include a second controller coupled to the probe and configured to regulate release of carbon dioxide from the carbon dioxide source to the bioreactor based at least in part on one or more measurements from the probe, and further, where the carbon dioxide is injected into the culture solution a carbon source enabling propagation of the at least one selected target species of microbe. Some embodiments include delivering at least a portion of the at least one target species of microbe to at least a portion of the at least one location, where at least a portion of the at least one target species of microbe being delivered comprises at least one live microbe. In some embodiments, the at least one live microbe is selected to be a well-adapted endemic species.

In some embodiments of the method, the water conditioning assembly includes an ozone generator coupled to an ozone contactor, where the ozone generator is configured to generate ozone and deliver the ozone to at least partially ozonate the supply water.

In some embodiments of the method, a solids filter is positioned upstream from an inlet of the ozone contactor.

In some embodiments of the method, a carbon filter and/or a UV light system are positioned immediately downstream from the solids filter, where the carbon filter and/or the UV light system are configured and arranged to at least partially de-ozonate the ozonated supply water. Further, at least one pressurized air supply is coupled to the bioreactor, where the at least one pressurized air supply can generate gas bubbles to at least partially aerate and/or agitate the culture solution in the bioreactor.

Some embodiments of the method further comprise delivering at least a portion of the at least one target species of microbe to at least a portion of the at least one location. In some embodiments, at least a portion of the at least one target species of microbe delivered comprises at least one live microbe. In some embodiments, the at least one live microbe is an endemic species of algae to the delivery location. In some further embodiments, the at least one live microbe is a live species selected to restore a normal soil flora mix of a cropland. In some other embodiments, the live species of algae is selected for its specific desired properties for improving the soil in the delivery location.

Some embodiments include a method comprising sampling the algal flora from an agricultural location, and selecting at least one desired algae species for propagation, where the at least one desired algae species is present in the agricultural location as an initial concentration. Some embodiments include propagating the at least one desired algae species in at least one bioreactor, and delivering the at least one desired species to the agricultural location to increase the concentration of the algae species to a concentration greater than the initial concentration.

In some embodiments of the method, the at least one bioreactor is adapted to propagate at least one desired species in a culture solution using in combination at least one of natural and artificial light, and at least one nutrient comprising at least a carbon source, where at least one desired species are freely suspended in and form part of the culture solution.

In some embodiments of the method, an algae nutrient supply is coupled to the at least one bioreactor and a controller for controlling flow between a water conditioning assembly and the at least one bioreactor. In some embodiments, the water conditioning assembly is coupled as an input of supply water to the at least one bioreactor to condition the supply water to a specified purity that enables substantially unhindered growth of the microalgae in the culture solution to a specified concentration. Further, in some embodiments, the controller is configured to control supply of the algae nutrient supply to the at least one bioreactor.

In some embodiments of the method, a carbon dioxide source coupled to the at least one bioreactor, where the carbon dioxide is injected into the culture solution as the carbon source. In some further embodiments of the method, a second controller is coupled to a probe, the second controller configured to regulate release of carbon dioxide from the carbon dioxide source to the bioreactor based at least in part on one or more measurements from the probe.

In some embodiments of the method, the water conditioning assembly includes an ozone generator coupled to an ozone contactor, where the ozone generator generates ozone and delivers the ozone to at least partially ozonate the supply water. In some further embodiments of the method, a solids filter is positioned upstream from an inlet of the ozone contactor.

In some embodiments of the method, the carbon filter and/or a UV light system are positioned downstream from the solids filter, where the at least one of the carbon filter and the UV light system at least partially de-ozonates the ozonated supply water. In some other embodiments of the method, the pressurized air supply is coupled to the bioreactor, where the pressurized air supply generates gas bubbles to at least partially aerate and/or agitate the culture solution in the at least one bioreactor.

### DESCRIPTION OF THE DRAWINGS

FIG. 1 depicts a first embodiment of the microalgae-based soil inoculating system of the invention.

FIG. 2 depicts a front-perspective view of a second embodiment of the microalgae-based soil inoculating system of the invention.

FIG. 3 depicts a side elevation view of a third embodiment of the microalgae-based soil inoculating system of the invention.

FIG. 4A depicts a field, five weeks after a crop of melons were planted and treated according to the method and with the system of the invention.

FIG. 4B depicts the same field of FIG. 4A at nine weeks after a crop of melons were planted and treated according to the method and with the system of the invention.

FIG. 5A depicts a melon plant in a section of field not treated according to the invention.

FIG. 5B depicts melon plants in a section of field treated according to the invention.

FIG. 6A depicts a melon growing in plant after nine weeks in a section of field not treated according to the invention.

FIG. 6B depicts a melon growing in plant after nine weeks in a section of field treated according to the invention.

FIG. 7 depicts a fourth embodiment of the microalgae-based soil inoculating system of the invention.

FIG. 8 depicts a fifth embodiment of the microalgae-based soil inoculating system of the invention.

FIG. 9 illustrates a soil enrichment system in accordance with some further embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives that fall within the scope of embodiments of the invention.

Some embodiments of the invention include a system capable of delivering a full range of micronutrients within microalgae to soil. In some embodiments, microalgae containing water (effluent) can be inoculated into soil thereby making the micronutrients immediately bioavailable to crops grown in the soil. In some embodiments, the system can be placed within an irrigation system between the water source and the water ports, through which irrigation water can be applied to crops. In some embodiments, the system can produce biofertilizers that are immediately bioavailable to crops, such that negligible runoff pollution occurs. Using this system, inorganic agricultural chemicals can be used more efficiently after being converted into a bioavailable form by the algae; therefore, the amount of chemicals needed is reduced.

In some embodiments, the system can be used to build soil organics with nutrient-rich algae biomass to recover depleted (nutrient poor) soils. In some embodiments, the system can facilitate and accelerate the transformation of a chemicals-based farm to an organic farm. In some embodiments, the system can deliver microalgae to the soil that dissolve soil carbonates, build polysaccharide content in the topsoil, and improve soil porosity up to 500% or more. In some embodiments, the system also provides for use of specific algal biotoxins in place of conventional chemical fungicides and other chemical poisons/toxins to manage nematodes and other harmful pests.

Some embodiments include a system that can comprise one or more bioreactors. In some embodiments, the system can comprise plural bioreactors. In some embodiments, when plural bioreactors are present, the bioreactors can be the same or different. Likewise, in some embodiments, the contents of the bioreactor can be the same or different. In some embodiments, the culture medium in a bioreactor of the system can comprise one or more types of microalgae. Some embodiments of the invention include those wherein: a) all of the microalgae are of the same type; b) two or more different types of microalgae are present; and/or c) one or more bioreactors contain one or more types of microalgae, and one or more other bioreactors contain one or more other types of microalgae.

In some embodiments, the microalgae in the bioreactor can propagate so an initial microalgae inoculant placed into the bioreactor can provide an endless supply of microalgae. In this instance, microalgae feed and water can be loaded into the bioreactor and a sufficient amount of microalgae biomass can be removed from the bioreactor periodically so as to keep the conditions within the bioreactor suitable for microalgae culture.

In some embodiments, the system and its method of use can improve overall crop production 5% to 30% or higher as compared to untreated crops. In some embodiments, the system and method of use can improve the texture, taste, size, nutrient content and/or yield of a crop as compared to untreated crop. In terms of agriculture use, in some embodiments, the system and its method of use can reduce total energy consumption, and/or reduce ecological pollution, and/or reduce greenhouse gas emission, and/or increase bioavailability of micronutrients and macronutrients, and/or reduce the use of chemical fertilizers, and/or reduce overall crop production cost, and/or reduce tillage cost, and/or reduce the need for and use of fungicides, herbicides and/or pesticides, and/or reduce soil compaction, and/or improve soil porosity, and/or increase microbial content of soil, and/or increase the organics content of soil, and/or reduce the amount of irrigation water needed to grow a crop, and/or reduce the occurrence of over fertilization, and/or reduce run-off and soil erosion, and/or improve plant characteristics and/or improve water/moisture retention by soil, all as compared to untreated crop and croplands.

In some embodiments, the system can be used to reduce or eliminate the buildup of carbonates in irrigation equipment by flowing microalgae-containing water through the irrigation equipment. In some embodiments, the system can also be used to reduce or eliminate buildup of carbonates in soil by inoculating the soil with microalgae-containing water.

Many different species and strains of microalgae can be used according to the crop needs. Algae may be collected and cultivated from the field where crops are to be grown or from commercial sources. Microalgae samples can be obtained from repositories at Arizona State University, University of California at Berkeley, University of Texas at Austin, Woods Hole Oceanographic Research Institute, Scripps Institute of Oceanography or other repositories.

Different species and strains of microalgae grow best under different conditions. The culture conditions within the bioreactor will be varied according to the particular species of microalgae present in the bioreactor. Conditions for culturing many different types of microalgae can be found in The Handbook of Microalgal Culture: Biotechnology and Applied Phycology (ed. Amos Richmond, Blackwell Publishing, Oxford, U. K., 2004), Algal Culturing Techniques: A Book for All Phycologists (ed. Robert A. Andersen, Elsevier Academic Press, 2005), and Microalgae: Biotechnology and Microbiology Cambridge Studies in Biotechnology (ed. E. W. Becker. Press Syndicate of the University of Cambridge, 1994), the disclosures of which are hereby incorporated in their entirety by reference.

In some embodiments, indigenous microalgae species can possess properties that make it optimal for growth under the environmental conditions of the target geographic location. In some embodiments, algae from non-indigenous locations or algal collections may be used to inoculate the soil of the target geographic location in order to maximize specific bioavailable compounds. Some embodiments include a method of inoculating soil that can comprise: obtaining a sample of soil from a target geographic location, and/or isolating a robust indigenous microalgae species from the sample, and/or culturing the microalgae to form a first inoculate. Further, the method can include inoculating a portable microalgae-based soil inoculating system with the first inoculate, and/or culturing the microalgae in the inoculating system to form a second inoculate, and/or inoculating soil of the target geographic location one or more times with the second inoculate. Further details are disclosed below.

In some embodiments, the system of the invention can employ various different types of water as the water source, including, but not limited to, wastewater, and/or well water, and/or lake water, and/or creek water, and/or pond water, and/or rainwater, and/or river water and/or freshwater. Since the water is intended for crop growth, it is preferred that the water source has low salinity and is free from heavy metals. In some embodiments, after exiting the micro-algae inoculating system, the inoculate-containing water can be delivered to a crop by any conventional irrigation means or system used in agriculture, for example, by flood, sprinklers or drip type of irrigation systems or by sprayer or aerial application. If applied by sprayer or aerial application, the treatment can be followed by sufficient water to drive the algae into the soil.

In some embodiments, the system and method can provide for continuous, semi-continuous, repeated or periodic treatment of soil with microalgae-containing inoculate. For example, in some embodiments, the soil can be treated with microalgae-containing inoculate daily, or every other day, or every third day, or semi-weekly, or every fourth day, or every fifth day, or every sixth day, or weekly, or biweekly, or every third week, or every fourth week, or monthly, or bimonthly, or quarterly, each trimester, or semiannually, or annually. In some embodiments, the soil can be treated with water not containing the microalgae and then with water containing microalgae inoculate, or vice versa. Some embodiments include a dilute, semi-concentrated and concentrated algal cultures with a single algal species or two or more different algal species. In some embodiments, the although it is optional, additional crop nutrients (macronutrients and/or micronutrients), aside from microalgae feed, can be included in the irrigation water. For example, in some embodiments, the nutrients such as calcium may be incorporated into the algal species for transport and uptake by the crops. The following table includes example macronutrients and micronutrients.

| | |
|---|---|
| Macronutrients | Micronutrients |

| | | | |
|---|---|---|---|
| Nitrogen | (N) | Boron | (B) |
| Phosphorus | (P) | Sulfur | (S) |
| Potassium | (K) | Copper | (Cu) |
| Carbon | (C) | Chloride | (Cl) |
| Oxygen | (O) | Iron | (Fe) |
| Magnesium | (Mg) | Molybdenum | (Mo) |
| Calcium | (Ca) | Manganese | (Mn) |
| | | Nickel | (Ni) |
| | | Zinc | (Zn) |
| | | Selenium | (Se) |
| | | Chromium | (Cr) |
| | | Cobalt | (Co) |
| | | Biotin | |
| | | Thiamin | |
| | | Vitamin B12 | |
| | | Vitamin B6 | |

Algae operate symbiotically with other organisms, both microorganisms and macro-organisms. While the primary object of the invention focuses on culturing algae, culturing algae in a diverse community of multiple microorganisms may offer useful solutions. Nitrogen-fixing microbes, called diazotrophs, fall into two main groups, free-living and symbiotic. Aerobic diazotrophs, of which there are over 50 genera, including Azotobacter, methane-oxidizing bacteria, and cyanobacteria, require oxygen for growth and fix nitrogen into soil when oxygen is present. Azotobacter, some related bacteria, and some cyanobacteria fix nitrogen in ordinary air, but most members of this group fix nitrogen only when the oxygen concentration is low. Aphanizomenon flosaquae reduces acetylene and fixes nitrogen in algal cultures. Some symbiotic bacteria belong to the genus Rhizobium such as Bradyrhizobium and Sinorhizobium, which colonize the roots of leguminous plants and stimulate the formation of nodules within which they fix nitrogen micro-aerobically. Green microalgae provide nitrogen, phosphorous, potassium, calcium and various other micronutrients. Accordingly, some embodiments include embodiments wherein one or more microalgae are co-cultured with or are inoculated into soil along with one or more diazotrophs.

In some embodiments, suitable microorganisms that can be co-cultured with or inoculated into soil along with the microalgae and/or algae can include actinomycetes, bacteria, fungi, and/or mycorrhizae. For example, some embodiments include actinomycetes, which are thread-like bacteria that look like fungi. While not as numerous as bacteria, they perform vital roles in the soil, where they help decompose organic matter into humus, which slowly releases nutrients. They also produce antibiotics to fight root diseases. The same antibiotics can be used to treat human diseases. Actinomycetes create the sweet, earthy smell of biologically active soil when a field is tilled.

Some embodiments can include the use of bacteria which can break down complex molecules and enable plants to take up nutrients. Some species release N, S, P and trace elements from organic matter. Others break down soil minerals and release K, P, Mg, Ca and Fe. Other species make and release natural plant growth hormones, which stimulate root growth. A few bacteria fix N in the roots of legumes while others fix N independently of plant association. Bacteria are responsible for converting N from ammonium to nitrate and back again depending on soil conditions. Various bacteria species increase the solubility of nutrients, improve soil structure, fight root diseases, and detoxify soil. In some embodiments, bacteria suitable for co-culture with the microalgae and for use in the system of the invention are disclosed in U.S. Pat. No. 7,736,508 to Limcaco (Jun. 15, 2010), the relevant disclosure of which is hereby incorporated by reference.

Some embodiments can include the use of fungi, some species of which can appear as thread-like colonies, while others are one-celled yeasts. Slime molds and mushrooms are also fungi. Many fungi aid plants by breaking down organic matter or by releasing nutrients from soil minerals. Fungi are generally early to colonize larger pieces of organic matter and begin the decomposition process. Some fungi produce plant hormones, while others produce antibiotics including penicillin. Several fungi species trap harmful plant-parasitic nematodes.

Some embodiments can include the use of mycorrhizae, a group of fungi that lives either on or in plant roots and act to extend the reach of root hairs into the soil. Mycorrhizae increase the uptake of water and nutrients especially in less fertile soils. Roots colonized by mycorrihizae are less likely to be penetrated by root-feeding nematodes since the pest cannot pierce the thick fungal network. Mycorrhizae also produce hormones and antibiotics, which enhance root growth and provide disease suppression. The fungi benefit from plant association by taking nutrients and carbohydrates from the plant roots where they live.

Aside from revitalization or nutrient supplementation of soil, some embodiments of the system and method can also be used in place of or to reduce the need for conventional herbicides, pesticides, fungicides and nematocides. For example, in some embodiments, after harvest, an algal species with specially selected toxins may be applied to manage nematodes and other soil predators. The algae with toxins are naturally occurring and typically die out after killing the nematodes. While it is possible for algae to mutate, indigenous algae will be far more robust and quickly crowd out any remaining toxic algae. Microalgae suitable for use as pesticides include algae from the genera Nostoc, Scytonema, and Hapalosiphon. Some embodiments can include the use of the system and methods in places such as soil-based farms, parks, hydroponic farms, aquaponics, nurseries, golf-courses, sporting fields, orchards, gardens, zoos and other such places where crops or plants are grown. Some embodiments can include the use of additional phytotoxins obtainable from microbes are described by Duke et al. (“Chemicals from Nature for Weed Management”, Weed Science, (2002) vol. 50, pg. 138-151). Some non-limiting example phytotoxins include actinonin, brefeldin, carbocyclic coformycin, cerulenin cochlioquinone, coronatine, 1,4-cineole, fischerellin, fumosin, fusicoccin, gabaculin, gostatin, grandinol, hydantocidin, leptospermone, phaseolotoxin, phosphinothricin, podophyllotoxin, prehelminthosporol, pyridazocidin, quassinoid, rhizobitoxin, tagetitoxin, sorgoleone syringotoxin, tentoxin, tricolorin A, thiolactomycin and usnic acid.

Some embodiments can include the use of a bioreactor adapted to receive and use natural and/or artificial light. As such, in some embodiments, the bioreactor can be adapted to permit exposure of microalgae to a light source. In some embodiments, the wall of the bioreactor can comprise a light-permeable material to permit exposure of the microalgae to light. If an artificial light source is used, the light source can be placed within or at the exterior of the bioreactor, e.g. according to U.S. Pat. No. 8,033,047, the entire disclosure of which is hereby incorporated by reference. Alternatively, in some embodiments, the system can comprise water conduit having through which microalgae-containing water in the bioreactor can be circulated to expose the microalgae to light. Some embodiments can include the use of a water conduit adapted to employ sunlight, reflected, bent, fiber optic or artificial light.

In some embodiments, the system can be run continuously, semi-continuously or in a batch-type operation.

In some embodiments, the system can further comprise one or more monitors or sensors adapted to monitor: a) growing conditions within the bioreactor; and/or b) microalgae cell titer/cell count in the water; and/or c) pH of the water; and/or d) salinity of the water; and/or e) the presence of undesired microbes in the bioreactor; and/or f) water level; and/or g) water pressure; and/or h) level of microalgae nutrients; and/or i) level of solids in the filtered water; and/or j) the level of undesired compounds in the water; and/or k) oxygen, ozone and/or CO₂ content in the water; and/or l) level of nitrogen compounds in the water; and/or m) clarity or opacity of the water; and/or n) level of desired compound(s) in the water; and/or o) water flow-rate; and/or p) weed algae; and/or q) algal predators; and/or) other contaminants.

In some embodiments, the monitor or sensors can be used to control operation of the system, such as by feedback regulation. In some embodiments, a monitor may generate one or more signals to controllers, which control the flow of materials into and/or out of the system. For example, in some embodiments, a microalgae cell titer monitor may send one or more signals to one or more flow controllers that the flow of source water or microalgae-containing water into and/or out of the system. In some embodiments, a pH monitor may send one or more signals to a CO₂ flow controller that controls the amount of, or rate at which, CO₂ is added to the system. In some further embodiments, a water level monitor may send one or more signals to a water flow controller that controls the amount of or rate of water flow into and/or out of the system. In some embodiments, a pH monitor may send one or more signals to an acid or base titrating unit that controls the amount of or rate of acid or base flowing into and/or out of the system.

In some embodiments, a water pressure monitor may send one or more signals to a water pressure regulator that controls the amount of or rate of water flow into and/or out of the system. In some embodiments, an ozone monitor may send one or more signals to an ozone flow controller that controls the amount of or rate at which ozone is added to the system. In some further embodiments, a clarity monitor may send one or more signals to a water clarity controller that controls the efficiency of filtration of water in the system. In some other embodiments, a nutrient monitor may send one or more signals to a nutrient source flow controller that controls the amount of or rate at which nutrient for the microalgae is added to the system.

In order to grow, plants and microalgae need nutrients such oxygen, carbon, nitrogen, phosphorus, potassium, magnesium, sulfur, boron, copper, chloride, iron, silicon, sodium, manganese, molybdenum, zinc, cobalt, vanadium, bismuth, iodine, water, carbon dioxide, air and/or others.

The profile of macronutrients and micronutrients provided by the microalgae will depend upon the strain or species of microalgae used. Plants may require a different spectrum of micronutrients and macronutrients during the different stages of the life cycle of the plant. Some embodiments provide a method of growing crops where the macronutrient and micronutrient profile of microalgae is matched with particular phases in the life cycle of a plant. In some embodiments, a field may receive regular nutrient feedings during crop growth and development with different species used depending on the needs of the crop. For example, microalgae A provides a nutrient profile A, microalgae B provides a nutrient profile B, and a target crop requires a nutrient profile A during the early stages of growth and a nutrient profile B ring of the latter stages of growth. In such a situation, the soil in which the crop is planted will be inoculated first with microalgae A during the early stages of growth of the target crop and will be inoculated then with microalgae B during the latter stages of growth of the target crop.

Some embodiments include a method of producing a crop comprising: planting a crop into soil and inoculating the soil with a first microalgae that provides a first nutrient profile; and/or allowing the plant to pass from a first stage of growth into a second stage of growth; and/or inoculating the soil with a second microalgae that provides a different second nutrient profile. In some embodiments, the first nutrient profile will be optimal for plant growth during the first stage, and the second nutrient profile will be optimal for plant growth during the second stage.

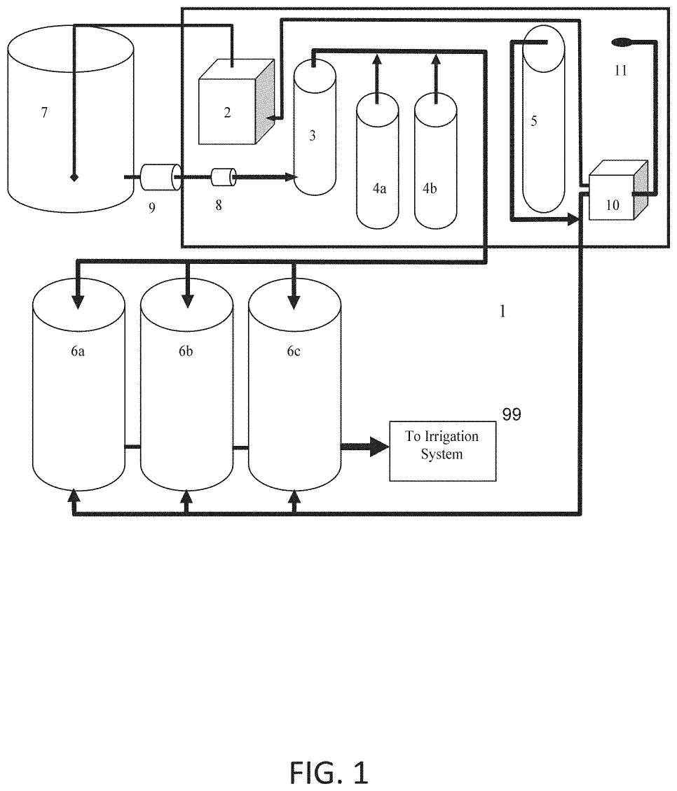

FIG. 1 depicts a first embodiment of a portable microalgae-based soil-inoculating system 1 of the invention. In some embodiments, the system comprises a water source 7, an ozone source 2, a carbon filter/UV light system, 3, a water pump 8, a solids filter 9, microalgae nutrient source 4a, 4b, bioreactors 6a, 6b, 6c, a carbon dioxide source 5, a pressurized air supply/air pump 10 and various and water conduits. In some embodiments, the pressurized air supply may be a blower, and/or air compressor, and/or rocker pump, and/or any other conventional producer or source of pressurized air. In some embodiments, air is taken from the atmosphere or a tank via the inlet 11, which optionally includes an air filter. In some embodiments, the air passes through the air pump 10 to an ozone source 2, whereby ozone-treated air is formed and conducted into a water source 7 to form ozone-treated water. In some embodiments, the air is also injected with a carbon dioxide source 5 to form carbon dioxide-treated air that is conducted into the bioreactors 6a-6c or into water entering the bioreactors. In some embodiments, the ozone treated water is filtered through a solids filter 9 a carbon filter and/or a UV light system 3 to form filtered water to which microalgae feed is added by the microalgae feed source 4a, 4b to form feed water, which is conducted into the bioreactor. In some embodiments, during initial startup, the bioreactors 6a, 6b, 6c are filled with water containing microalgae nutrients and are then inoculated with a first inoculate containing microalgae. In some embodiments, the carbon dioxide-containing air is injected into the microalgae-containing water in the bioreactors 6a, 6b, 6c. In some embodiments, the water in the bioreactors 6a, 6b, 6c is recirculated for a period of time until the microalgae cell titer/cell count has reached a target level suitable for use as an inoculant. In some embodiments, the water from the system 1 is then flowed into irrigation water to form a microalgae-containing inoculate as the effluent, which is applied to the soil from an irrigation system 99.

Various different operation parameters can be controlled. For example, in some embodiments, one or more heaters are optionally included in the system to heat water conducted through the system and/or heat the culture medium in the bioreactor, thereby permitting culture of microalgae and use of the system even during cold weather.

In some embodiments, the volume of system water and its flow rate into the irrigation water of the irrigation system 99 can be adjusted as needed to provide the appropriate level of inoculation and water penetration into the soil. For example, in some embodiments, a 200-acre field might receive a total daily volume of 500 to 1 thousand gallons of water at a delivery rate of about 21 gallons/hour to 42 gallons/hour. In some embodiments, the inoculate obtained from the bioreactor (e.g., such as one or more of the bioreactors 6a, 6b, 6c) can be applied to soil with or without further dilution. For example, in some embodiments, the system 1 can be operated such that all water used for irrigation flows through the bioreactor. Otherwise, in some embodiments, the system 1 can be operated such that the inoculate, the effluent of the bioreactors 6a, 6b, 6c, is diluted with additional irrigation water prior to application to the soil.

In some embodiments, the microalgae cell titer (the cell count) in a bioreactor fluctuates over time; therefore, the cell titer of the effluent varies as well. The titer provides important metrics regarding the unit's health and productivity. Generally, the titer in the effluent can be at least 1,000,000 cells per ml up to 30,000,000 cells per ml. The titer is also species specific, and can be higher or lower than the range stated above.

In some embodiments, the ozone can be used to destroy unwanted microbes present in the irrigation water prior to entering the bioreactor. Any organic contaminants present in the system can be removed by ozonolysis as described in U.S. Pat. Nos. 5,947,057 and 5,732,654 to Perez et al. Organic contaminants include herbicides, pesticides, and fungicides among other things. In some embodiments, the ozone source can be an ozone generator. Ozone generators may include the model 01 by Pacific Ozone, the Nano by Absolute Ozone, and the OZ8PC20 by Ozotech. In some embodiments, the water is treated with ozone as required according to the quality of the water entering the system. In some embodiments, the concentration of ozone in the water and prior to filtration through a carbon filter will vary with water quality but have an ozone level sufficient to sterilize the water. In some embodiments, the treatment of the water with ozone may be improved by employing a mixer that mixes the water and ozone.

In some embodiments, the carbon filters and UV light systems are used to remove ozone from the irrigation water prior to entering the bioreactor. In some embodiments, the carbon filter generally employs a minimum of 0.75 ft³ of activated carbon. In some embodiments, the carbon filter and UV light systems are flow-through systems. In some embodiments, the suitable carbon filters include the 0.75 ft³ “Upflow Carbon Filter System” from Affordable Water (www.affordablewater.us). UV systems may include the “CSL Series” by Aquafine, and the “UVS3XX Series” by UV Sciences (www.aquaneuv.com; Valencia, Calif.). In some embodiments, the UV light system can be used to disinfect water prior to entering the bioreactor, and/or to destroy ozone, destroy chlorine or chloramines prior to entering the bioreactor. In some embodiments, the UV light system can disinfect by inactivating or killing microorganisms in the water.

In some embodiments, when a solids filter is present, it can be used to remove solids from the irrigation water prior to entering the bioreactor. In some embodiments, the solids filter can be a flow-through filter. In some embodiments, suitable solids and filters can include the “X100” bag filter from www.filterbag.com or the “FV1” bag filter from www.aquaticeco.com.

In some embodiments, suitable carbon filters and/or solids filters can include, but not be limited to, media filters, disk filters, screen filters, microporous ceramic filters, carbon-block resin filters, membrane filters, ion-exchange filters, microporous media filters, reverse osmosis filters, slow-sand filter beds, rapid-sand filter beds, cloth filters, and/or any other conventional filter.

In some embodiments, carbon dioxide can be used as a carbon source for microalgae. In some embodiments, the carbon dioxide can be added directly or indirectly to the bioreactor. In some embodiments, carbon dioxide source can be a tank containing carbon dioxide, a carbon dioxide generator, a carbon dioxide sequestering device that sequesters carbon dioxide from the atmosphere, or a combination thereof. Alternatively, carbon dioxide captured from air can be used, e.g. U.S. Pat. No. 8,083,836, the entire disclosure of which is hereby incorporated by reference. In other embodiments, the carbon dioxide can be sourced from acetic acid and/or calcium carbonate.

Atmospheric air contains approximately 0.035-0.04% wt., of carbon dioxide. While atmospheric air can serve as a source of carbon dioxide for the microalgae, the concentration of carbon dioxide is generally too low to sustain the rapid proliferation of microalgae in the bioreactor. Accordingly, in some embodiments, carbon dioxide can be added to the air that is fed into the culture medium. In some embodiments, the concentration of carbon dioxide in the air added to the culture medium can be generally in the range of about 1-3% wt., 1.5-2.5% wt., 1.8-2.2% wt., or about 2% wt.

In some embodiments, a water pump can be included in the system. In some embodiments, when present, the water pump can facilitate the flow of water through the water conduits and/or bioreactors of the system. In some embodiments, if a water pump is not included, the pressure of the irrigation water entering can be sufficient to drive water through the system.

In some embodiments, an air pump or blower (the terms are used interchangeably herein) can be included in the system. In some embodiments, air pump can facilitate the flow of air, which may or may not include carbon dioxide or ozone, through the air conduits, water source and/or bioreactors of the system.

The size or operating capacity of each piece of equipment comprising the system can be varied as needed. For example, in some embodiments, a portable system comprising a total bioreactor capacity of 500 gallons of culture medium can support 200 acres of land and will generally require the following minimum operating capacities for the indicated components: a) ozone source-1.5 g/hr; (dry air); b) solids filter-40 g/min maximum flow with a minimum 2 ft² surface area; c) carbon filter-0.75 ft³ minimum; d) water pump-10 gal/min minimum; e) air pressurized air supply/air pump-25 cfm at 60″ H₂0 minimum; f) microalgae feed source-1.0×10⁶ cells/ml minimum; g) liquid carbon dioxide source-80 l/week.

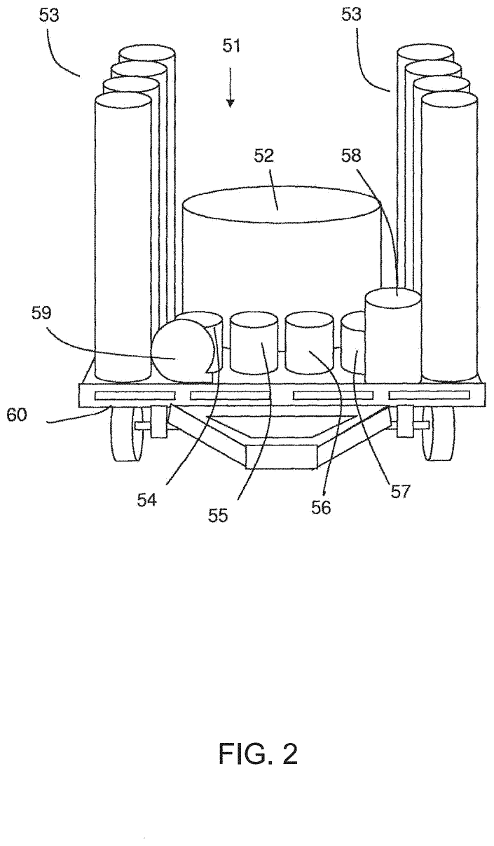

FIG. 2 depicts another embodiment comprising a portable system 51, where the components of the system 51 are mounted on a trailer. In some embodiments, the system 51 comprises a water tank 52, a plurality of bioreactors 53, an ozone generator 54, a clarifier 55, a combination filter/UV light system 56, nutrient feed supply 57, CO₂ source 58, a pressurized air supply 59 and a trailer 60. As shown, any of the water tank 52, plurality of bioreactors 53, ozone generator 54, clarifier 55, combination filter/UV light system 56, nutrient feed supply 57, CO₂ source 58, and pressurized air supply 59 can be mounted onto the trailer 60.

In some embodiments, the system 51 can accommodate a flow-through capacity of about 0.35-0.7 gal/min and can be used to support a field in the range of 200-1000 acres. In some embodiments, the water tank 52 can receive water from the on-site water source of a farm. In some embodiments, the system 51 can comprise eight bioreactors (500-gallon total capacity), a water tank, air filter, solids filter, carbon filter, UV light system, ozone source, carbon dioxide source, microalgae nutrient source, pressurized air supply and water pump (not shown). In some embodiments, the bioreactors 53 can have light-permeable walls such that sunlight is used as the light source. In some embodiments, carbon dioxide and air can be bubbled into the lower part of the bioreactor 53 so the bubbles agitate the culture medium as they rise. In some embodiments, the system 51 optionally comprises a mechanical agitator. In some embodiments, the system 51 can provide a minimum of about 800,000 microalgae cells per second via the effluent, assuming a water flow rate of about 0.35 gal/min.

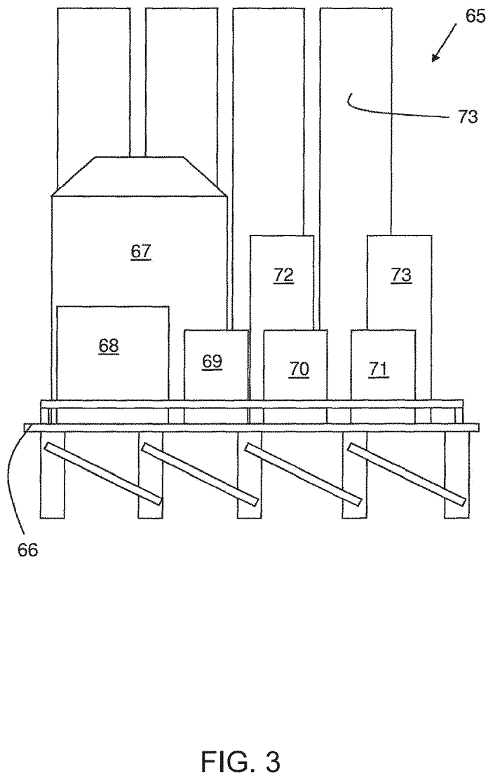

FIG. 3 depicts a side elevation view of another system 65 of the invention comprising an elevated portable platform 66, water tank 67, pressurized air supply 68, ozone source 69, clarifier 70, water filter 71, nutrient source 72, carbon dioxide source 73 and bioreactors 74. In some embodiments, one or more components can be mounted on the platform and one or more components can be placed on the ground or onto one or more other platforms.

Although FIGS. 2 and 3 depict a water tank 52, 67 as the water supply, in other embodiments, a flowing water source can be used instead; therefore, in some embodiments, the system of the invention optionally includes one or more water tanks as the water supply or excludes a water tank as the water supply. Although not depicted in FIGS. 2 and 3, in some embodiments, the effluent of one or more bioreactors can be fed into the water flow of an irrigation system. In some embodiments, the systems described herein can be placed within a partial or full enclosure even though the systems are portable.

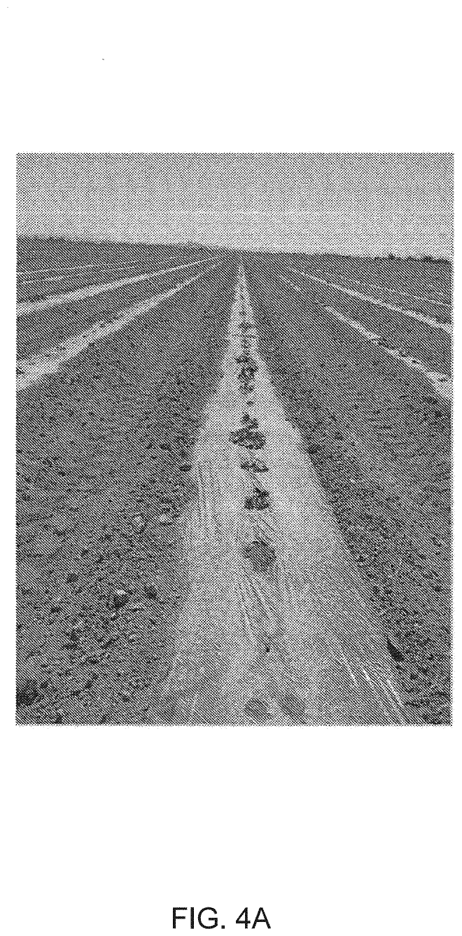

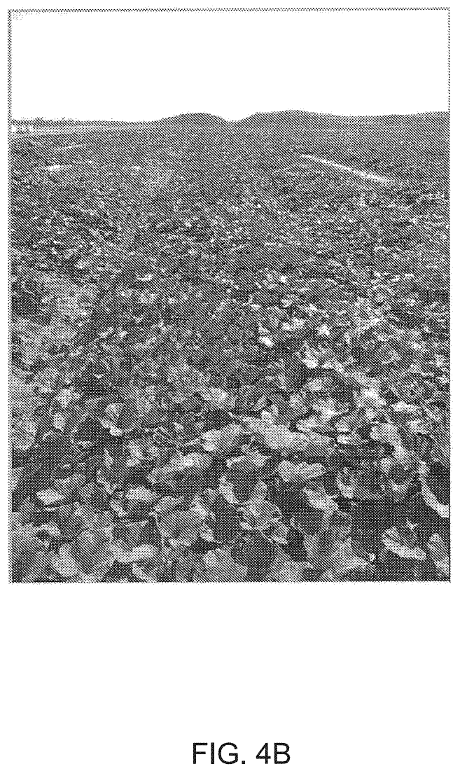

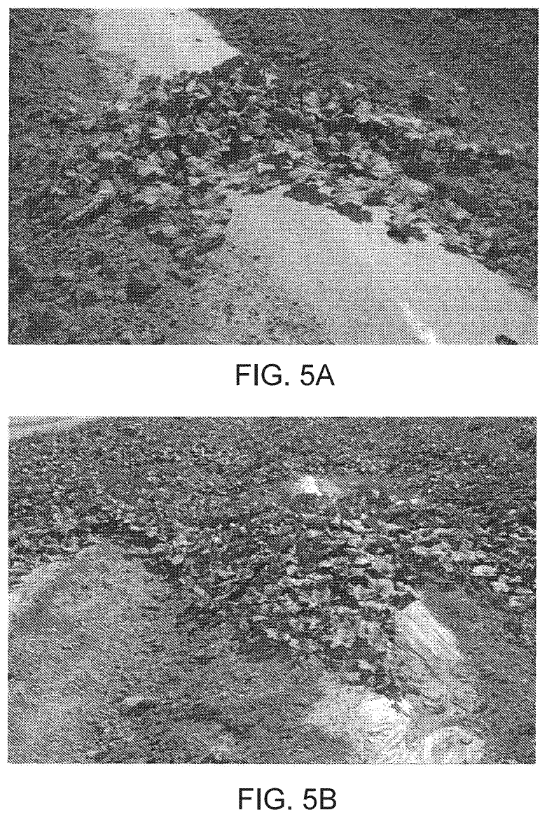

In some embodiments, the performance of the system of FIG. 2 was evaluated in a crop study where melon crops were planted in 200 acres of land. The land was divided into control and sample sections (e.g., see FIGS. 5A-5B). The control sections only received irrigation water and were not treated with microalgae supplement. The sample sections received only irrigation water containing the microalgae supplement. Melon seeds were planted before irrigating with the algae supplement in the soil. The control plants were irrigated about every fourth day, depending on the heat. The sample plants were irrigated on the same schedule as the controls. Various aspects of plant and fruit growth were evaluated five weeks (shown in FIG. 4A) and nine weeks (shown in FIG. 4B) after planting.

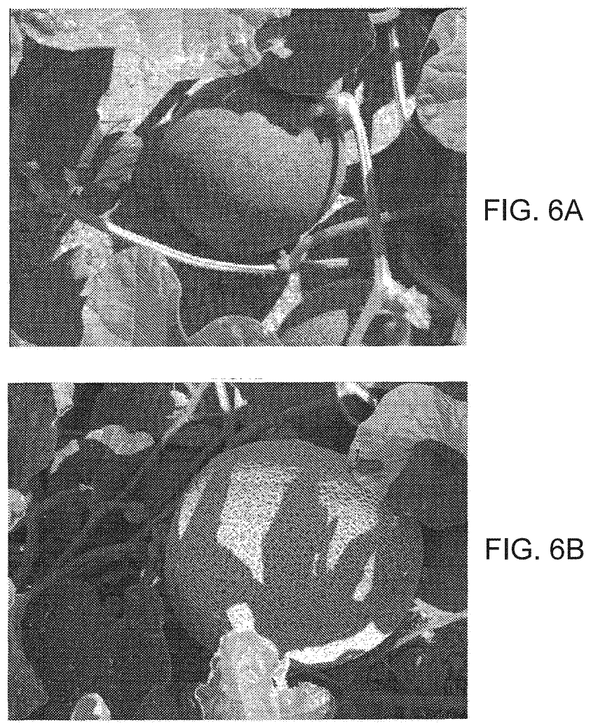

Briefly, the crop grown according to the systems and methods disclosed herein produced larger and hardier plants. For example, compare FIG. 5A (showing a control plant) to FIG. 5B (showing a sample plant). Further, compare the larger melons of FIG. 6B to the control plant shown in FIG. 6A. Moreover, the sample plants produced more flowers per vine, had improved fruit texture and taste, improved sugar content, improved nutritional content, improved appearance, and improved Vitamin A content. The specific details and results are described in Example 1.

In some embodiments, the system can further comprise one or more monitoring devices for performing functions, including, but not limited to, measuring CO₂ flow rate, CO₂ content in the culture, O₂ content in the culture, pH, cell density and temperature in the culture, measuring macronutrient content in the culture or effluent, measuring micronutrient content in the culture or effluent, or measuring the microalgae titer in the culture or effluent.

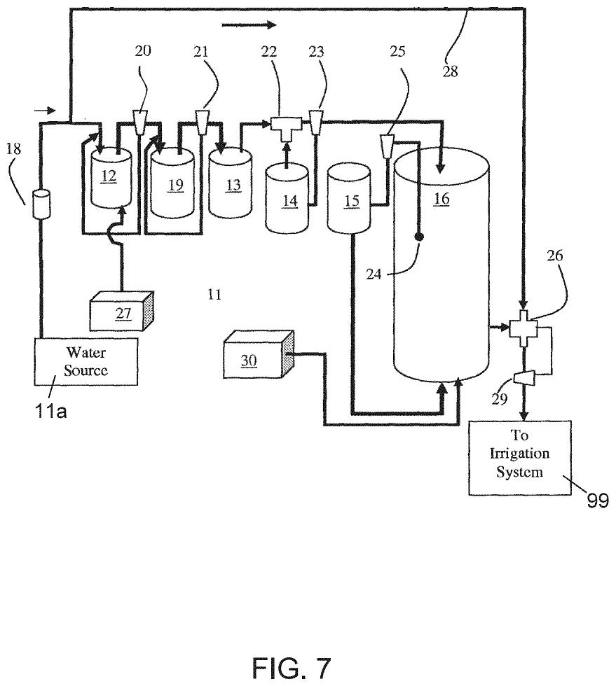

FIG. 7 depicts an alternate embodiment of the system of the invention. In some embodiments, the system 11 is suitable for low, medium and high-volume irrigation applications. In some embodiments, the system 11 comprises an optional pump 18 adapted to receive water from a pressurized or unpressurized water source 11a. In some embodiments, the water received from the water source 11a is ozonated within an ozone contactor 12 that receives ozone from an ozone generator 27 and conducted to a clarifier/filter 19 that removes precipitated solids from the water. In some embodiments, after clarification, the water is conducted to a carbon filter or UV light system 13, that removes the ozone, and through to a mixer 22 that mixes the water with algae feed material obtained from the algae nutrient supply 14. In some embodiments, the algae/water mixture is mixed by use of air bubbles, which are produced by a pressurized air supply 30, which conducts air to an air diffuser in the base of the bioreactor 16. In some embodiments, the water containing nutrient material is conducted into the bioreactor 16, wherein microalgae are cultured. In some embodiments, the effluent containing the microalgae exits the bioreactor 16 and passes through a valve 26 that regulates the ratio of flow of water between the by-pass water source line 28 and the bioreactor effluent. In some embodiments, the controller 29 controls the valve 26 to achieve the desired ratio of volume of flow between untreated source water (from by-pass line 28) and the effluent to provide an inoculant containing a desired or target microalgae titer.

In some embodiments, the system 11 can include one or more different controllers. For example, in some embodiments, the controller 20 can comprise an optional feedback loop where water that has been improperly ozonated can be fed back into the ozone contactor 12 for proper treatment. In some embodiments, the controller 21 can comprise an optional feedback loop such that water that has been insufficiently clarified can be fed back into the clarifier 19 for proper clarification. In some embodiments, the controller 23 can provide control over the algae nutrient supply 14 to regulate the amount of feed material that is charged into the water. In some embodiments, the controller 25, by use of a pH probe 24, can provide control over the carbon dioxide source 15 that charges carbon dioxide into the bioreactor 16 to regulate the concentration of carbon dioxide in the water and ensure the water has the proper carbon dioxide concentration. In some embodiments, the algae/water mixture can be mixed by use of air bubbles, which are produced by a pressurized air supply 30, which conducts air to an air diffuser in the base of the bioreactor.

In some embodiments, the system 11 can comprise a portable platform (or body or frame, not shown) onto which plural components of the system are mounted. In some embodiments, the each of the individual components of the system can be individually replaceable. Although the components are indicated as single components, each of the components can be present in plurality independently of other components of the system.

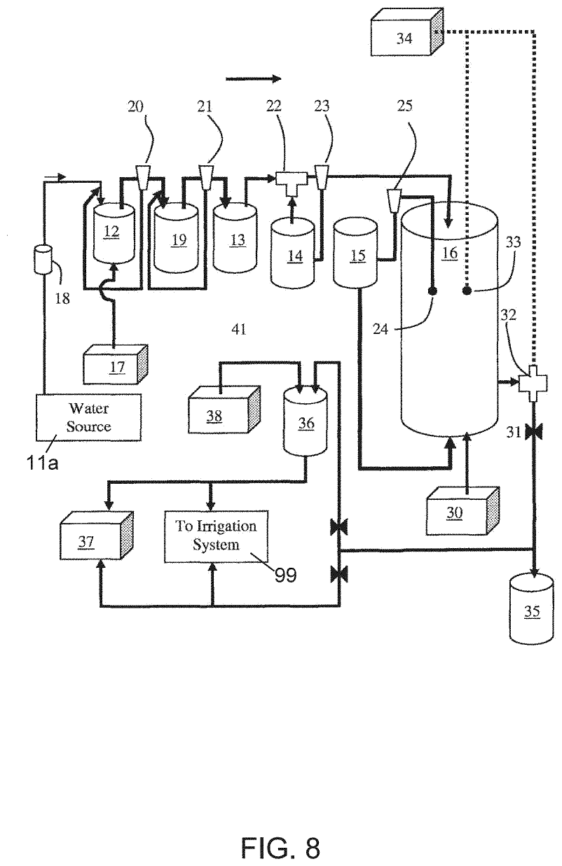

FIG. 8 depicts an alternate embodiment of the system of the invention. In some embodiments, the system 41 as shown can be suitable for low, medium and high-volume irrigation applications or flowing to a distribution tank 37. In some embodiments, the distribution tank 37 may sit on a trailer for portability. In some embodiments, the system 41 comprises an optional pump 18 adapted to receive water from a pressurized or unpressurized water source 11a. In some embodiments, the water from the water source 11a is ozonated within an ozone contactor 12 that receives ozone from an ozone generator 17. In some embodiments, the ozonated water is conducted to a clarifier/filter 19 that removes precipitated solids from the water. In some embodiments, after clarification, the water is conducted to a carbon filter or UV light system 13, that removes the ozone, and through to a mixer 22 that mixes the water with algae fertilizer/additives obtained from the algae nutrient supply 14. In some embodiments, the water containing nutrient material can be conducted into the bioreactor 16, where microalgae can be cultured. In some embodiments, the algae/water mixture can be mixed by use of air bubbles, which are produced by the pressurized air supply 30, which conducts air to an air diffuser in the base of the bioreactor as discussed earlier with respect to the system 11 of FIG. 7. In some embodiments, one or more probes 33 can be placed in the culture to measure the critical parameters including pH, temperature, cell density, water mixing velocity, dissolved gasses and nutrients. In some embodiments, an optional telemetry device 34 can send the metrics from the probes (monitoring devices or controllers) to a computer server for remote monitoring. In some embodiments, an optional telemetry capable microscope can assist the remote culture monitoring. In some embodiments, the optional telemetry device 34 comprises the optional telemetry capable microscope.

As used herein, telemetry device 34 can be any device capable of facilitating communication between the system of the invention and a communications and/or control center remote from or at a different geographic locale than the system of the invention. In some embodiments, the telemetry device 34 can employ any type of wireless communication system and can employ any frequency of light waves, radio waves, sound waves, infrared waves, hypersonic waves, ultraviolet waves, other such wavelengths/frequencies and combinations thereof. In some embodiments, the telemetry device 34 employ an IP network (such as the Internet), GSM (global system for mobile communications) network, SMS (short message service) network, other such systems and combinations thereof.

In some embodiments, a flow imaging device 32 can create images of the algae, predators and contaminants in the culture for quality control (QC) purposes, and can send this data to the telemetry device 34. In some embodiments, the effluent containing the microalgae can exit the bioreactor and pass through a valve 31 that regulates the flow of the bioreactor effluent. In some embodiments, the optional dewatering device 35 can concentrate the algae into slurry of the desired density, which may flow to irrigation or portable containers 37. In some embodiments, an optional microorganism mixer 36 can enable the user to blend the final product with, in addition to algae, beneficial bacteria, fungi or other organisms 38 that work symbiotically with algae.

In some embodiments, the system 41 can include one or more different controllers. In some embodiments, the controller 20 can comprise an optional feedback loop such that water that has been improperly ozonated can be fed back into the ozone contactor 12 for proper treatment. In some embodiments, the controller 21 comprises an optional feedback loop such that water that has been insufficiently clarified can be fed back into the clarifier 19 for proper clarification. In some embodiments, the controller 23 provides control over the algae nutrient supply 14 in order to regulate the amount of feed material that is charged into the water. In some embodiments, the controller 25, by use of a pH probe 24, can provide control over the carbon dioxide source 15 that charges carbon dioxide into the bioreactor in order to regulate the concentration of carbon dioxide in the water and ensure the water has the proper carbon dioxide concentration. In some embodiments, the system 11 can comprise a portable platform (or body or frame, not shown) onto which plural components of the system are mounted. In some embodiments, each of the individual components of the system can be individually replaceable. Although the components are indicated as single components, each of the components can be present in plurality independently of other components of the system.

In some embodiments, a system similar to the system 41 of FIG. 8 can be used to reclaim degraded or abandoned soil. In some embodiments, an algae and microorganism mixture produced by the system may be applied though irrigation or spaying on the soil surface to restore vital nutrients. Algae and the other microorganisms continue to flourish in the soil as long as soil moisture is available. Algae deliver micronutrients, attract other microorganisms and add organic matter (humus) to the soil. In some embodiments, the process can rehabilitate degraded or abandoned soil.

In some further embodiments, a system similar to the system 41 of FIG. 8 can culture other microorganisms in the same culture or separate containers for blending before the culture flows into the irrigation or portable containers.

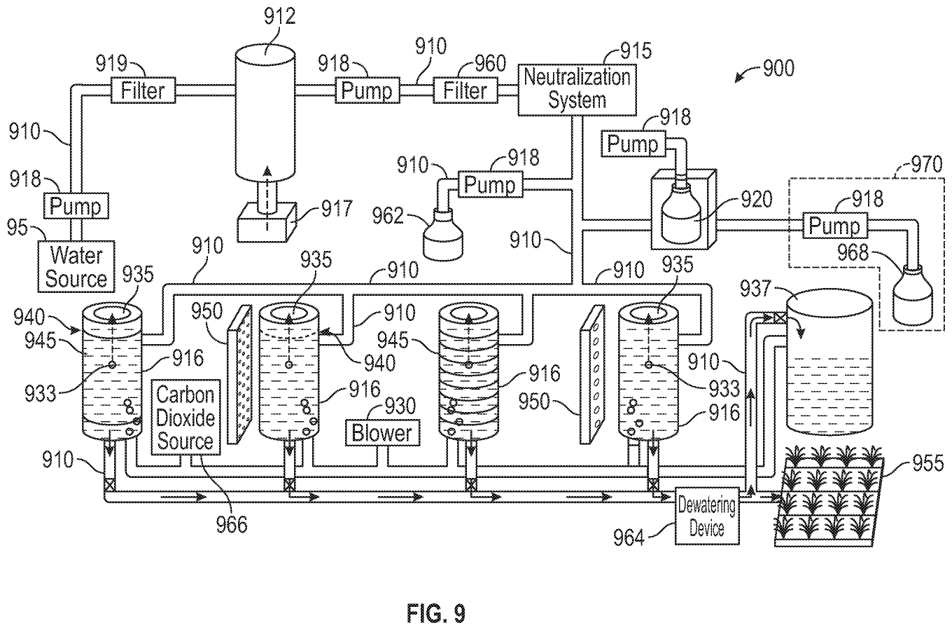

FIG. 9 illustrates a soil enrichment system 900 in accordance with some further embodiments of the invention. Some embodiments include a solids filter 919, a water storage tank 912, a sterilization system 917, and a neutralization system 915. A growth priming system may comprise one or more nutrient solution feeds, such as first and second nutrient solution containers 920, 962 to add nutrient solutions to the treated water. A bioreactor system may comprise one or more bioreactors 916 to facilitate inoculation with and growth of the microorganism. The systems and methods may include various additional systems and subsystems, such as one or more nutrient solution containers, refrigerators, light sources, blowers (e.g., at least one pressurized air supply), carbon dioxide sources, pumps, valves, fluid conduits, air conduits, gas conduits, air filters, gas filters, control systems, sensors, air conditioning units, exhaust systems, portable housings, and/or exterior holding tanks.

In some embodiments, one or more pumps 918, such as peristaltic pumps, may propel irrigation water from the water source 95 through fluid conduits 910. The water source 95 supplies water to the soil enrichment system 900. Water flowing from the water source 95 may be referred to as “irrigation water.” The water source 95 may comprise any suitable source of irrigation water appropriate for irrigation of plants. In some embodiments, the water source 95 may be under pressure, such as water from a well or a public utility in a city, town, or municipality. In some embodiments, the water source 95 may be substantially unpressurized. For example, the water source 5 may comprise a stationary water reservoir, reclaimed wastewater, well water, lake water, creek water, pond water, rainwater, river water, and/or freshwater.

Some embodiments of the soil enrichment system 900 may comprise an automated cleaning system 970 controlled by a control system. The automated cleaning system 970 may comprise a cleaning solution container 968 for holding the cleaning solution and a pump 918 for pumping the cleaning solution from the cleaning solution container 968 into the fluid conduit and/or the one or more bioreactors 916. In some embodiments, each of the one or more bioreactors 916 may comprise a dedicated valve for connection of a fluid conduit leading to the cleaning solution container 968 for the cleaning solution.

In some embodiments, one or more bioreactors 916 may be inoculated with the microorganism inoculant by any suitable method, such as manual inoculation through a port 935 in the bioreactor 916. In some further embodiments, the neutralized irrigation water containing nutrient solution may be conducted into any one or more of the bioreactors 916 until it reaches a preselected fill level 940.

In some embodiments, a light source 945/950 may be configured to project light onto and/or into each of the one or more bioreactors 916. In some embodiments, the light source 945/950 may comprise LED lights in any suitable configuration to provide light to the microorganism culture. For example, in one embodiment, a first light source 945 may be positioned within the one or more bioreactors 916. In another embodiment, the first light source 945 may overlay an exterior surface of the one or more bioreactors 16. In another embodiment, a second light source 950 may be outside of and adjacent to an exterior surface of the one or more bioreactors 916.

In some embodiments, a control system suitable for implementing one or more of the present embodiments may include a computer system communicatively linked to a PLC system 934. The PLC system 934 may be communicatively linked to the one or more sensors 933 and may provide measurements obtained by the one or more sensors 933 to a processor and/or database for remote monitoring, remote data access, and/or remote control of the soil enrichment system 900. The PLC system 934 may similarly be communicatively linked and configured to control pumps 918, valves, sterilization system 917, neutralization system 915, at least one pressurized air supply 930, lights 950, and/or any carbon dioxide source.

In some embodiments, a carbon dioxide source 966 can be used to supply a carbon source to the microorganism culture. Carbon dioxide may be added directly and/or indirectly to the one or more bioreactors. The carbon dioxide source 966 may be a tank containing carbon dioxide gas, a carbon dioxide generator, a carbon dioxide-sequester for sequestering and temporarily storing atmospheric carbon dioxide or a combination thereof.

In some embodiments, the microorganism culture may be released from the one or more bioreactors 916 through the outlets, flow through the one or more fluid conduits, and flow into the external holding tank 937 for storage. In various embodiments, the external holding tank 937 may comprise an at least partially transparent material such as high or low-density polyethylene, polycarbonate, acrylic, and/or PVC to allow natural or artificial light to penetrate through the external holding tank 937 and into the microorganism culture. In some embodiments, the external holding tank 937 may comprise a sterile aeration system to support the health of the microorganism culture. In some embodiments, the external holding tank 937 may comprise a cone-shaped base to ensure complete drainage of the microorganism culture when it is released onto a target field 955.

In some embodiments, the exterior holding tank 937 may comprise a cooling system such as a refrigerator to cool the microorganism culture during storage. The refrigerated exterior holding tank may be configured to receive the microorganism culture and/or microorganism slurry, maintain its sterility, and store it at any suitable temperature.

In some embodiments, the dewatering device 964 may be configured to deliver the concentrated microorganism slurry to the target field 955 and/or the exterior holding tank 937. The dewatering device 964 may concentrate the microorganism culture through any suitable process such as, but not limited to: 1) flocculation and sedimentation; 2) flotation and collection; and/or 3) centrifugation. Further details and operational characteristics of the soil enrichment system 900 are described in U.S. patent application Ser. No. 15/647,005, the entire contents of which are incorporated by reference.

Some embodiments include methods of isolation, selection, and use of endemic microbes for agriculture production areas using any of the systems described herein. For example, some embodiments of the invention include methods of selecting, collecting, and growing algae for delivery to an agricultural production area. Specifically, in some embodiments, the methods focus on collecting, isolating, and/or propagating endemic microbes, primarily algae, for mass delivery to the same biome from which the algae was collected. In some embodiments, the agricultural production area comprising the biome may be a farm field, and/or a raised bed, and/or a greenhouse, and/or a golf course, and/or degraded land, and/or an indoor growing facility. Some further embodiments include collecting, isolating, and/or propagating, and delivering other endemic microbes in addition to, or separately from algae. For example, some embodiments include collecting, isolating, and/or propagating, and delivering a bacterial species. Other embodiments include collecting, isolating, and/or propagating, and delivering a fungal species.

In some embodiments of the invention, the algae may be delivered through a variety of means including, but not limited to, canal irrigation, flood irrigation, and/or drip irrigation, and/or various conventional overhead spray techniques, and/or various conventional hydroponic cultivation techniques. In some embodiments of the invention, the effects of delivering algae to the agricultural production area may be an increase in soil organic matter, and/or improvement in soil structure, and/or reduction in water and fertilizer utilization, and/or increase in crop yield and the nutrient value of the product, and/or an overall improvement in soil health, and/or reduction in water and chemical runoff, and/or an increase in carbon dioxide sequestered from the air by the soil.

Some embodiments of the invention include a method of obtaining a soil and/or water sample from an agricultural production area, and/or culturing microbes from the soil sample, and/or selecting a desirable species from the soil sample, and/or propagating the selected desirable species in greater numbers and concentration, and/or delivering live microbes back to the agricultural production area (e.g., such as dispersing the live microbes in solution over a soil area of a farm, or biome area).

The following steps constitute a non-limiting embodiment of a method for collecting, selecting, and propagating endemic algae from an agricultural production area (e.g., such as a farm or other plant propagation facility):

Some embodiments include a step of collecting one or more quantities of soil from one or more locations on the agricultural production area. In some embodiments, each quantity or a total quantity of collected soil can be about 100 grams. In some other embodiments, the quantity can be less than 100 grams or more than 100 grams.

Some embodiments include a step of collecting one or more quantities of water from one or more locations on the agricultural production area (e.g., such as from a surface water source). In some embodiments, each quantity or a total quantity of collected water can be about 50 grams. In some other embodiments, the quantity can be less than 50 grams or more than 50 grams. In some other embodiments, at least some of the water can be collected from a sub-surface source, a run-off source, or a spring or well source.

In some embodiments, one or more of the water and/or the soil quantities can be refrigerated to 35° F. to 40° F. prior to subsequent processing locations, including, without limitation, a laboratory or facility.

In some embodiments, about 10 grams of soil or 10 ml of water from each sample can be added to a 100 ml culture jar containing 75 ml of AF6 (Watanabe) media. In some embodiments, more or less soil and/or water can be added to the culture jar. In some further embodiments, more or less AF6 (Watanabe) media can be used. In some embodiments, the soil and/or water can be incubated in the culture jar. In some embodiments, the incubation can occur overnight while being exposed to a 100 to 200 PAR light source. In some embodiments, the light source can comprise or emit wavelengths of about 450 nm to 485 nm and/or about 625 nm to 740 nm. In some embodiments, exposure can be approximately 12 to 24 hours per day.

In some embodiments, a portion of the incubated samples can be propagated in Agar-coated petri dishes. For example, in one non-limiting embodiment, samples can be plated-out onto four 100×15 mm petri dishes with AF6 agar with 10 μl samples with loop sterilization in-between each streak to dilute the sample. In some embodiments of the invention, the petri dishes can be at least partially closed (e.g., taped to 75% closed) and placed upside down in front of a 100 to 200 PAR light source for one to two weeks. In some embodiments, the light source can comprise or emit wavelengths of about 450 nm to 485 nm and about 625 nm to 740 nm. In some embodiments, exposure can be about 12 to 24 hours per day.

In some embodiments, when isolated axenic algae colonies have grown to a specific size, the algae colonies can be harvested aseptically, and placed into a sterile test tube with sterile AF6 media. For example, in some embodiments, when isolated axenic algae colonies have grown to about 3 mm in diameter, the algae colonies can be harvested aseptically and placed into a sterile test tube with sterile AF6 media.

Some embodiments can include an incubation time of one to two weeks, followed by selecting the tubes with the highest biomass. In some embodiments, the incubation can occur while being exposed to a 100 to 200 PAR light source. In some further embodiments, the light source can contain wavelengths of about 450 nm to 485 nm and about 625 nm to 740 nm. In some embodiments, exposure can be about 12 to 24 hours per day. In some further embodiments, temperatures can range between about 70° F. and 80° F.

Some embodiments include sub-culturing each tube into a new tube, followed by placing the contents of the original tube into a sterile 500 ml bottle with AF6 media outfitted with sterile air injection. In some embodiments, the sub-culturing tubes can be exposed to a 100 to 200 PAR light source. In some embodiments, the light source can contain wavelengths of about 450 nm to 485 nm and 625 nm to 740 nm. In some embodiments, exposure can be about 12 to 24 hours per day.

Some embodiments include incubating the bottle for 3-5 days, and selecting the bottles with the fastest growth rate and highest biomass, and identifying with a new strain ID. In some embodiments, the incubation can occur while being exposed to a 100 to 200 PAR light source. In some embodiments, the light source can contain wavelengths of about 450 nm to 485 nm and about 625 nm to 740 nm. In some embodiments, exposure can be about 12 to 24 hours per day. In some embodiments, temperatures can range between about 70° F. and 80° F.

In some embodiments of the invention, the strain IDs of the incubated samples can be recorded in the strain ID database with date time and location of collection along with any additional algal characteristics. Further, in some embodiments, new test tubes can be inoculated with each newly identified strain and place in algal library.

In some embodiments of the invention, a further step can include an artificial selection process to improve, growth rate, maximum density and other desired characteristics. In some embodiments, the artificial selection process can contain algae strains that are exposed to preferred culture conditions. In some embodiments, algae strains that have an improved growth rate, higher maximum density, or other desired characteristics can be selected over the inferior strains for future use. In some embodiments, inferior algae strains may be put through the artificial selection process to further improve the growth rate, maximum density or other desired characteristics.

In some embodiments of the invention, one or more the steps can be performed in a laboratory or facility that is remote from the agricultural production area. In some embodiments of the invention, one or more the steps can be performed in a laboratory or facility that is proximate to or part of the agricultural production area. In some embodiments, all of the steps can be performed in the same location. In other embodiments, at least some of the steps can be performed in one location, and one or more other steps can be performed in another location.

In view of the above description and the examples below, one of ordinary skill in the art will be able to practice the invention as claimed without undue experimentation. The foregoing will be better understood with reference to the following examples. All references made to these examples are for the purposes of illustration. The following examples should not be considered exhaustive, but merely illustrative of only a few of the many embodiments contemplated by the present invention.

### Example 1

### Evaluation of the System for Melon Growth

The system of the invention was used to grow the Yosemite variety of cantaloupe melons. About 200 acres were infused with microalgae-containing irrigation water. The crop was watered every five days during afternoons due to high ambient temperatures (120° F.). Microalgae were added to the irrigation water continuously with each watering. Algae from the phylum Chlorophyta and Cyanophyta were added to the irrigation water at a combined density of 6 billion cells per minute. The algae were cultured in media shown in the table below.

FW Media

| | | |
|---|---|---|
| Final Conc. | Stock Solutions | Use Rate |
| (g/L) | (g/L) | (ml/L) |

N & P Solution

| | | | |
|---|---|---|---|
| NaNO₃ | 0.344 | 34.4 | 10 |
| KCl | 0.303 | 30.3 | |
| NaH₂PO₄ | 0.03 | 2.91 | |

Missing element solutions

| | | | |
|---|---|---|---|
| CaCl₂—2H₂O | 0.11 | 11 | 10 |
| MgSO₄•7H₂O | 0.246 | 24.6 | |

Trace Element Solution

| | | | |
|---|---|---|---|
| Na2EDTA-2H₂O | 0.0045 | 4.5000 | 1 |
| FeCl₃•6H₂O | 0.00289 | 2.8910 | |
| MnCl₂—4H₂O | 0.00098 | 0.9800 | |
| ZnSO₄•7H₂O | 0.000036 | 0.0360 | |
| CoCl₂•6H₂O | 0.000011 | 0.0110 | |
| Na2MoO₄—2H₂O | 0.00012 | 0.1200 | |
| CrO₃ | 0.000075 | 0.0750 | |
| SeO₂ | 0.000005 | 0.0050 | |
| CuSO₄•5H₂O | 0.000012 | 0.0120 | |

Vitamins

| | | | |
|---|---|---|---|
| Biotin | 0.000025 | 0.025 | 1 |
| Thiamine HCl | 0.0000175 | 0.017 | |
| B12 | 0.000015 | 0.015 | |

The melons were harvested and the following observations were made when comparing melons grown according to the invention to melons not grown according to the invention.

| Metric | Description |
|---|---|
| Productivity | Improved melon production 20% by weight. |
| Size | Fruit increased in diameter by 22%. |
| Texture | Texture of meat of the fruit held or improved. |
| Shelf-life | The shelf-life was extended by 4 days. |
| Taste | Taste of the fruit held or improved. |
| Sugar | Sweetness of the fruit improved by 20%. |
| Appearance | Appearance, color, of the fruit held or improved. |
| Vitamin A | Vitamin content improved by 20%. |

Various different dimensions of the melon plants were measured at 9-weeks after planting both for control plants and plants grown with the system of the invention. The observed dimensions are detailed below.

| | | | |
|---|---|---|---|
| Parameter | Control | Sample | Fold increase |

| | | | | | |
|---|---|---|---|---|---|
| Trunk Diameter | 0.129 | in | 0.38 | in | 2.9 |
| Stem diameter | 0.05 | in | 0.125 | in | 2.5 |
| Average Leaf length | 2.5 | in | 4 | in | 1.6 |
| Largest Leaf length | 3.5 | in | 7 | in | 2.0 |
| Overall plant radius | 37.8 | in | 87.12 | in | 2.3 |
| Overall plant height | 5.7 | in | 15 | in | 2.6 |
| Flower size width | 0.9 | in | 2.3 | in | 2.6 |
| Melon diameter | 2.3 | in | 5.5 | in | 2.4 |

The algae infused melon fields required 50% less N inorganic fertilizer and 40% less P and K. Micronutrient savings were on the order of 70%. The farmer reported a 5-fold improvement in soil porosity, looseness, which enabled deeper crop roots. Higher soil porosity also enabled symbiotic macro and microorganisms to enter the field such as earthworms. The farmer reported that the melon fields needed over 50% less pesticide application, because the algae infused crops seemed to make their own biopesticides that discouraged invaders, such as white flies that destroyed neighboring fields. The farmer used 70% less fungicide as the algae enabled longer roots that were more resistant to nematodes and other soil pests. Accordingly, the system of the invention provides substantial improvements in characteristics of plants and fruits grown with the system of the invention.

### Example 2

### Crop Growth Employing Two Different Microalgae

Prior to planting the seeds of a crop in soil, the soil is irrigated repeatedly with an inoculate containing a first species from the phylum Chlorophyta of microalgae until the soil has achieved the desired properties of increased organics with polysaccharides in the soil to increase water retention Seeds are planted in the treated soil and irrigated repeatedly with an inoculate containing a different second species from the phylum Cyanophyta of microalgae to infuse the soil with nitrogen sequestered from the atmosphere until the crop has reached maturity. The crop is then harvested using known methods. At this point a third species also from the phylum Cyanophyta is introduced into the irrigation water and delivered to the soil where it produces a biological toxin to kill unwanted pests in the soil. The first species of the phylum Chlorophyta of microalgae is used to enhance the fertility and other properties of the soil by increasing the organics in the soil which enhances the colonization by other micro and macro organisms which further enhance the soil by converting nutrients into forms more available to the crop and by increasing the porosity of the soil. The second species from the phylum Cyanophyta of microalgae is used to add nitrogen to the soil thereby reducing the amount of nitrogen fertilizer needed by the crop. The third species from the phylum Cyanophyta is used to eliminate or reduce the number of pests in the soil.

### Example 3

### System Employing Co-Culture of Two Different Microalgae

A system containing a co-culture of two different microalgae strains are prepared by preparing a culture medium in one or more bioreactors and inoculating it with one or more blue-green algae (cyanobacteria or Cyanophyta) and one or more green algae (Chlorophyta). Both algae can be independently unicellular or colonial; however, unicellular species are preferred. Some Chlorophyta include those of the class Chlorophyceae, which includes those of the order Chaetopeltidales, Chaetophorales, Chlamydomonadales, Chlorococcales, Chlorocystidales, Dunaliella, Microsporales, Oedogoniales, Phaeophilales, Sphaeropleales, Tetrasporales or Volvocales. Some Chlorophyta species include Chlorella fusca, Chlorella zofingiensis, Chlorella spp., Chlorococcum citriforme, Chlorella stigmataphora, Chlorella vulgaris, Chlorella pyrenoidosa and others. Some Cyanophyta include those of the order Chroococcales, Gloeobaterales, Nostocales, Oscillatoriales, Pseudanabaenales, and Synechococcales. The algae are co-cultured with natural and/or artificial light. The titer of algae in the culture medium is allowed to increase to a target level of about 1 MM to 100 MM cells per ml. The culture medium is discharged from the bioreactor and mixed in with water for irrigation.

As used herein and unless otherwise specified, the term “about” or “approximately” are taken to mean +−0.10%, +−0.5%, +−0.2.5% or. +−0.1% of a specified valued. As used herein and unless otherwise specified, the term “substantially” is taken to mean “to a large degree”, “at least a majority of”, greater than 70%, greater than 85%, greater than 90%, greater than 95%, greater than 98% or greater than 99%.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A culturing system comprising:
a bioreactor adapted to propagate microalgae in a culture solution using in combination at least one of natural and artificial light, and at least one nutrient comprising at least a carbon source, wherein the microalgae are freely suspended in and form part of the culture solution; and
a water conditioning assembly;
an algae nutrient supply coupled to the bioreactor; a first controller configured for controlling fluid flow between the water conditioning assembly and the bioreactor, the water conditioning assembly coupled as an input of supply water to the bioreactor, and configured to condition the supply water to a specified purity that enables substantially unhindered growth of the microalgae in the culture solution to a specified microalgae concentration, and wherein the first controller is configured to control delivery of the algae nutrient supply to the bioreactor; and a carbon dioxide source coupled to the bioreactor, wherein the carbon dioxide is injected into the culture solution as the carbon source.

2. The system of claim 1, further comprising a second controller coupled to a probe, the second controller configured to regulate release of carbon dioxide from the carbon dioxide source to the bioreactor based at least in part on one or more measurements from the probe.

3. The system of claim 2, wherein the probe is a pH probe configured to measure a pH of the culture solution.

4. The system of claim 1, wherein the water conditioning assembly includes an ozone generator coupled to an ozone contactor, wherein the ozone generator is configured to generate ozone and deliver the ozone to at least partially ozonate the supply water.

5. The system of claim 4, further comprising a solids filter upstream from the ozone contactor.

6. The system of claim 5, further comprising a carbon filter and/or a UV light system positioned downstream from the solids filter, wherein the at least one of the carbon filter and the UV light system are configured and arranged to at least partially de-ozonate the ozonated supply water.

7. The system of claim 1, further comprising at least one pressurized air supply coupled to the bioreactor, wherein the at least one pressurized air supply is configured to generate gas bubbles to at least partially aerate and/or agitate the culture solution.

8. The system of claim 7, wherein the gas bubbles include at least one of CO₂, N₂, and O₂.

9. The system of claim 1, further comprising at least one water reservoir or tank providing or coupled to the input of supply water.

10. The system of claim 1, further comprising a mobile trailer supporting at least the bioreactor, the water conditioning assembly, and the carbon dioxide source.

11. The system of claim 1, wherein the microalgae algae nutrient supply comprises at least one of a fertilizer, a macro-nutrient, a micro-nutrient, and at least two different microalgae species; and
wherein the macro-nutrient is selected from the group consisting of phosphorus, nitrogen, carbon, silicon, calcium salt, magnesium salt, sodium salt, potassium salt, and sulfur; and the one or more micronutrients is selected from the group consisting of manganese, copper, zinc, cobalt, molybdenum, vitamins and trace elements; and
wherein the micro-nutrient comprises at least one of a vitamin and a mineral added to the conditioned supply water.

12. The system of claim 1, further comprising a telemetry system configured for at least one of remote monitoring and controlling operation of one or more of the first controller, the second controller, the bioreactor, and at least one component or assembly of the water conditioning assembly.

13. The system of claim 1, wherein the artificial light comprises LED lights positioned at least one of within the bioreactor and/or proximate to an exterior surface of the bioreactor and exposing the microalgae to light.

14. The system of claim 1, wherein the carbon dioxide source comprises at least one of a tank comprising carbon dioxide liquid or gas, a carbon dioxide generator, and a carbon dioxide-sequester that sequesters and temporarily stores atmospheric carbon dioxide.

15. The system of claim 1, wherein the algae supply comprises at least one of a first algae type, and a second algae type.

16. The system of claim 1, further comprising a flow-imaging device coupled to an output of the bioreactor, the flow imaging device configured to create images of at least one of algae, predators, and contaminants in the culture solution for quality control monitoring.

17. The system of claim 1, further comprising a microorganism mixer configured to blend at least one of algae, bacteria, viruses, and fungi with any of the culture solution exiting the bioreactor.

18. A method comprising:
preparing one or more microbe-containing samples from at least one location of a current or planned plant growth area; preparing at least one cultured sample by culturing microbes from the sample;
selecting at least one target species of microbe from the at least one cultured sample;
propagating the at least one selected target species of microbe to increase the concentration of the at least one target species of microbe in the at least one cultured sample by:
providing a bioreactor adapted to propagate the at least one selected target species in a culture solution, the at least one selected target species being freely suspended in and forming part of the culture solution; coupling a feed source to the bioreactor and a first controller for controlling flow between a water conditioning assembly and the bioreactor, the water conditioning assembly coupled as an input of supply water to the bioreactor to condition the supply water to a specified purity that enables substantially unhindered growth of the at least one selected target species in the culture solution to a specified concentration, and wherein the first controller controls supply of the feed source to the bioreactor; and providing a carbon dioxide source coupled to the bioreactor, and regulating the release of carbon dioxide from the carbon dioxide source to the bioreactor, wherein the carbon dioxide is injected into the culture solution as a carbon source enabling propagation of the at least one selected target species of microbe.

19. The method of claim 18, further comprising a second controller coupled to a probe and the bioreactor, wherein the second controller regulates the release of carbon dioxide from the carbon dioxide source to the bioreactor.

20. The method of claim 18, further comprising delivering at least a portion of the at least one target species of microbe to at least a portion of the at least one location.

21. The method of claim 20, wherein at least a portion of the at least one target species of microbe being delivered comprises at least one live microbe.

22. The method of claim 21, wherein the at least one live microbe is an endemic species of algae to the delivery location.

23. The method of claim 21, wherein the at least one live microbe is a live species selected to restore a normal soil flora mix of a cropland.

24. The method of claim 23, wherein the live species of algae is selected for its specific desired properties for improving the soil in the delivery location.

25. The method of claim 18, wherein the water conditioning assembly includes an ozone generator coupled to an ozone contactor, wherein the ozone generator generates ozone and delivers the ozone to at least partially ozonate the supply water.

26. The method of claim 25, further comprising positioning a solids filter upstream from the ozone contactor.

27. The method of claim 26, wherein at least one of a carbon filter and/or a UV light system are positioned downstream from the solids filter, wherein the at least one of the carbon filter and the UV light system at least partially de-ozonates the ozonated supply water; and at least one pressurized air supply coupled to the bioreactor, wherein the at least one pressurized air supply generates gas bubbles to at least partially aerate and/or agitate the culture solution in the bioreactor.

28. A method comprising: sampling algal flora from an agricultural location; selecting at least one desired algae species for propagation from the algal flora, the at least one desired algae species being present in the algal flora of the agricultural location at an initial concentration; propagating the at least one desired algae species in at least one bioreactor; and delivering the at least one desired species to the agricultural location to increase the concentration of the algae species to a concentration greater than the initial concentration.

29. The method of claim 28, wherein the at least one bioreactor is adapted to propagate the at least one desired algae species in a culture solution using in combination at least one of natural and artificial light, and at least one nutrient comprising at least a carbon source, wherein at least one desired algae species are freely suspended in and form part of the culture solution; and an algae nutrient supply coupled to the at least one bioreactor and a controller for controlling flow between a water conditioning assembly and the at least one bioreactor, the water conditioning assembly coupled as an input of supply water to the at least one bioreactor, and conditions the supply water to a specified purity that enables substantially unhindered growth of the at least one desired algae species in the culture solution to a specified concentration, and the controller controls supply of the algae nutrient supply to the at least one bioreactor; and
a carbon dioxide source coupled to the at least one bioreactor, wherein the carbon dioxide is injected into the culture solution as the carbon source.

30. The method of claim 29, further comprising a second controller coupled to a probe, wherein the second controller regulates release of carbon dioxide from the carbon dioxide source to the bioreactor based at least in part on one or more measurements from the probe.

31. The method of claim 29, wherein the water conditioning assembly includes an ozone generator coupled to an ozone contactor, wherein the ozone generator generates ozone and delivers the ozone to at least partially ozonate the supply water.

32. The method of claim 29, wherein a solids filter is positioned upstream from the ozone contactor, wherein the solids filter removes solids from supply water.

33. The method of claim 29, wherein at least one of a carbon filter and/or a UV light system are positioned downstream from the solids filter, wherein the at least one of the carbon filter and the UV light system at least partially de-ozonates the ozonated supply water.

34. The method of claim 33, wherein at least one pressurized air supply is coupled to the bioreactor, wherein the at least one pressurized air supply generates gas bubbles to at least partially aerate and/or agitate the culture solution in the at least one bioreactor.
